(19) United States
(12) Patent Application Publication
(10) Pub. No.: US 2009/0254193 A1
(43) Pub. Date: Oct. 8, 2009

(54) Medical Prosthesis Implant Casting Process

(75) Inventor: Guillaume Kerboul, Sheffield (GB)

Correspondence Address: DRINKER BIDDLE & REATH (DC), 1500 K STREET, N.W., SUITE 1100, WASHINGTON, DC, 20005-1209, US

(73) Assignee: DONCASTERS MEDICAL TECHNOLOGIES LIMITED, Melbourne (GB)

(21) Appl. No.: 12/083,507

(22) PCT Filed: Oct. 27, 2006
(86) PCT No.: PCT/GB2006/004014

§ 371 Date: Apr. 11, 2008

(30) Foreign Application Priority Data
Nov. 1, 2005 (GB) 0522278.1

(51) Int. Cl. A61F 2/36; B22C 9/00; B22C 9/04; B22C 9/12

(52) U.S. Cl. 623/23.15; 164/15; 164/35

(57) ABSTRACT

A method of casting a hollow hip prosthesis head comprises forming a wax pattern of a femoral head including two cores. The first is a central socket-forming core and the second a ceramic hollow-forming core. The wax pattern is coated with a ceramic slurry to form a shell in which core extensions are captivated. The wax is melted and evacuated and the shell is fired to form a mould. Metal is cast and, when cool, the shell and cores are removed and each aperture formed in the head by the hollow-forming core is sealed.

### BACKGROUND

The present invention relates to replacement joint and other structural medical implants, in particular femoral head replacement implants. Currently preferred hip prostheses include a femoral head having a socket for connection to a stem, which stem is fixed in or to the patient's femur. An acetabular ball cup may or may not be provided for insertion into the patient's pelvis and adapted to receive in a close sliding fit the new artificial head.

To be suitable for use in a medical device, a material must exhibit the appropriate functional properties, mainly mechanical properties, for the particular application and must be biocompatible. Biocompatibility is the ability of a material to perform with an appropriate host response in a particular application. For example, in the complex environment of the human body, metal alloys are subject to electrochemical corrosion with the bodily fluids acting as an electrolyte and, to be biocompatible, a metal alloy used in an implantable medical device must exhibit very low corrosion over the projected lifetime of the device. Metal particles released by corrosion may be concentrated locally or distributed systemically and it is important that the type and amount of material released does not pose a danger to the patient. Cobalt-chromium based alloys, developed for the aerospace industry, are used in many medical device applications, including implantable medical devices, because of their strength, corrosion resistance, and biocompatibility. For example, cobalt-chromium alloys, typified by alloys conforming to ASTM standard specifications, such as, ASTM F-75-01, STANDARD SPECIFICATION FOR COBALT-28 CHROMIUM-6 MOLYBDENUM ALLOY CASTINGS AND CASTING ALLOY FOR SURGICAL IMPLANTS, and ASTM-799, STANDARD SPECIFICATION FOR COBALT-28 CHROMIUM-6 MOLYBDENUM ALLOY FORGING FOR SURGICAL IMPLANTS, are often used as components of modular prosthetic devices such as prosthetic hip and knee joints.

A prosthetic joint typically includes paired load bearing surfaces, commonly comprising a first surface of a metal alloy component paired with a second surface comprising a metal, a polymer, a ceramic, bone, or bone cement. When load bearing surfaces move relative to each other, such as during articulation of a prosthetic joint, friction can cause the surfaces to spall. The wear debris, known medically as third bodies, originating from the load bearing surfaces of an implanted medical device can initiate a histiocytic reaction in which the body's immune system is activated to release enzymes to dissolve the particles of debris. However, because the wear debris is usually a relatively hard material, such as a metal or polycarbon compound, the enzymes either fail to dissolve the debris or dissolve the debris only with the passage of considerable time. On the other hand, the enzymes do react with tissue and bone and may weaken or dissolve the bone supporting or adjacent to the medical device. In the case of a prosthetic joint, weakening of the bone or osteolysis may shorten the life of the device and may eventually render the supporting bone unusable. Further, surface erosion can eventually lead to failure of the load-bearing surfaces, requiring replacement or repair of the surfaces. In the case of implanted medical devices, replacement or repair entails expensive and risky surgery.

Typically, where a polymer cup is employed, this is in the form of a lining of a metal cup that is inserted into a patient's acetabulum. As a consequence, the femoral ball is necessarily smaller in size, since the metal cup must be of limited magnitude in order to fit into the patient's acetabulum. This means that the area of the mating surfaces of the head and polymer cup is also limited, increasing the pressures between them, and hence the wear. WO-A-2005/070344 describes a method of providing a smooth hard surface of a medical implant and if, this can be achieved, then a metal-to-metal interface can be permitted since the wear between is consequently less. And in that event, the area of the mating surfaces can be maximised which further reduces the wear between them.

However, if the head is solid, it is very heavy and that is undesirable for several reasons. A presently available design provides a hollow head cast in two parts: a hollow, hemispherical head, and a base connected to the head. The base is provided with a cup socket to permit connection to the femoral component. Between them, the base and head require considerable machining in order to permit interconnection of them by ion beam welding. However, it would be desirable to provide a hollow head that did not require machining, nor indeed, welding. Both these processes add significantly to the cost of the final product and also complicate traceability of the manufactured product. Often, the production (by casting) of the individual components may be carried out separately from any subsequent machining and/or welding. Consequently, there are more opportunities for inadequate accounting for the different stages of the production of the final product.

In any event, the welding of the base to the rim of the hemispherical head results in substantial heating of the hemispherical surface near the rim, and this may adversely impact the crystal microstructure near the rim which could influence the wear and or smoothness of the hemispherical surface.

U.S. Pat. No. 6,129,764 provides a relatively hollow (humeral) head with an open base to receive a variable connector to the humerus. SU-A-619179 provides a hollow cap for fitting to a reshaped femoral head. EP-A-375600 discloses forged femoral components welded together. DE-A-3907530 discloses a hollow femoral head formed from sheet material, and provided with a neck cup by welding.

CH-A-274021 discloses a method of making a hollow prosthesis comprising making a destructible core from a first material and casting a second destructible material as shell around the core in a mould, removing the first destructible core leaving the shell of the second destructible material and forming a ceramic mould having the second destructible material shell as its void and casting metal therein to form a hollow metal prosthesis.

It is an object of the present invention to provide a novel process of construction of a medical prosthesis head, and a head so constructed.

### BRIEF SUMMARY OF THE DISCLOSURE

In accordance with the present invention there is provided a method of casting a hollow medical prosthesis head comprising the steps of:
forming a pattern of the head, said pattern comprising a domed surface and a trunnion-seat-forming base, and a gate extension;
disposing a hollow-forming core in the head and an extension of said core through the base;
coating the pattern and said extension with a shell-forming slurry in which said core extension is captivated;
curing said shell to form a mould;
casting metal into the mould so as to form an aperture through the base of said head around said core extension;
when cool, removing said shell and at least said core extension; and
closing the or each aperture.

Preferably, said closure is effected by weld-filling of said aperture(s), although it could be closed by a plug. Preferably, said weld-filling (or plug) uses the same metal as the cast metal.

The pattern is preferably moulded from a settable fluid material such as wax or a thermoplastics material. In the case of wax, said curing step includes the step of re-melting the wax and draining the melted wax from the shell once the shell has cured to the extent that it retains its shape during said draining. Subsequently, if the shell is a ceramic mix, it may be fired to complete the curing process.

Preferably, there are a plurality of said extensions and apertures formed thereby, preferably four. They may be round in section but may also be elongate in a circumferential direction for reasons explained further below.

Preferably, said pattern includes a trunnion-forming part on said base and a trunnion is thereby integrally formed on the head during said casting step. A socket-forming core may be disposed in said trunnion-forming part, said socket-forming core including an extension also captivated by said shell-forming slurry.

Both the socket-forming core and hollow-forming cores may be ceramic. The hollow-forming core may toroidal in shape, whereby a supporting column for the trunnion is formed. Indeed, the trunnion may be formed by the eye of said toroid, and the socket-forming core may extend into the eye.

Said gate extension is conveniently off said domed surface.

A method as claimed in any preceding claim, wherein all of said core is removed from the head prior to said closure. The hollow void left by removal of said hollow-forming core may be evacuated and/or filled with an inert gas before said closure is effected.

The hollow-forming core is removed by shattering the core with a vibrating tool. Alternatively, it may be chemically etched to remove it. For either purpose, said extensions may usefully be elongate in section in order to facilitate access to the outside and removal of debris. A suitable dissolving agent is an alkaline solution, such as potassium hydroxide, that does not damage the cast metal.

The cast metal may be a cobalt/chromium/molybdenum alloy.

Said moulding of the pattern may comprise the steps of:
positioning said core or cores in a pattern-forming mould by connection of said extension or extensions to a base of said mould;
closing said mould with a domed cover; and
injecting curable fluid material the mould.

Said domed cover may conveniently have a gate aperture forming said gate extension and said injection of said fluid material may be through said gate aperture.

The method is particularly suitable when said hollow medical prosthesis head is a hip prosthesis head. Nevertheless, although described herein in relation to, and being particularly suitable for, hip replacement prostheses, the present invention is not limited thereto and the principles could be employed in other prostheses such as shoulder joints.

In another aspect of the present invention, a medical prosthesis head comprises a casting of metal provided with a domed surface and a base, and having an integrally cast hollow void, an integrally cast trunnion seat, and closed core pin aperture or apertures in the base of the head, said aperture(s) being of smaller dimensions than the void.

By “smaller dimensions” is meant that the core cannot be withdrawn through the aperture or apertures after casting but have either to be left behind, which is a perfectly feasible option, or be fluidised so as to flow out of one or more of the apertures, whether under the effects of gravity, vibration-spillage or pressurised ejection. In any event, while the apertures are desirably large for this purpose, they are also desirably small to reduce the effects of subsequently having to close them afterwards.

The prosthesis head may have an integrally cast trunnion on said trunnion seat. This also is preferred in order to reduce post-casting operations. Indeed, said trunnion preferably includes an integrally cast socket. The trunnion is advantageously supported by an integrally cast column spanning said void and connected with said domed surface.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### BRIEF DESCRIPTION OF THE DRAWINGS

An embodiment of the present invention is further described hereinafter, by way of example, with reference to the accompanying drawings, in which:

FIG. 1 is a side section through a human hip region showing a hip prosthesis of the type to which the present invention relates:

FIG. 2 is a section through a wax pattern casting mould, with cores in place, partly in section;

FIG. 3 is a section through a ceramic shell incorporating the wax pattern prior to metal casting;

FIG. 4 is a perspective view of a cast prosthesis prior to complete removal of all core material and gate; and,

FIG. 5 is a section through an alternative form of head also in accordance with the present invention.

### DETAILED DESCRIPTION

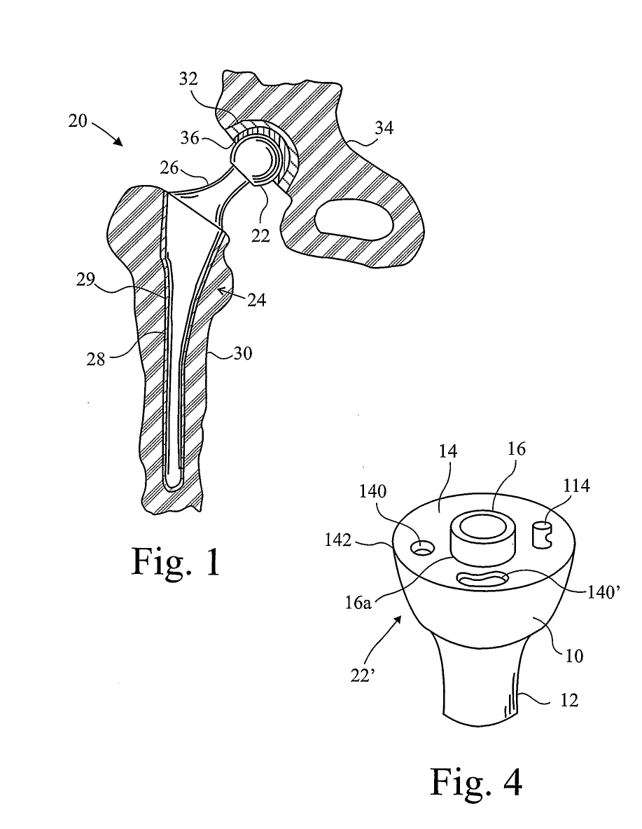

Referring in detail to the drawings where similar parts of the invention are identified by like reference numerals, and, more particularly to FIG. 1, an artificial prosthetic hip joint 20 includes a pair of interfacing load bearing surfaces arranged to move relative to each other. The prosthetic hip joint 20 typically includes a ball 22 that is connected to a body 24 comprising a neck 26 and a stem 28. The stem 28 may be held in place in the femur 30 by a variety of methods, including the use of cementing agents 29, an interference fit, a threaded attachment mechanism, or biological fixation.

A cup-shaped socket 32 is anchored in the pelvis 34 by any of a variety of known techniques, such as cementing; press fitting; the use of screws; the use of a textured, knurled, or threaded exterior; the use of a biological fixation mechanism or by a combination of biological and mechanical fixation. The ball 22 is positioned adjacent to the concave surface of the socket 32. A socket insert 36, commonly comprising a polymer, such as an ultra-high molecular weight polyethylene (UHMWPE) or an ultra-high molecular weight, cross linked polyethylene (UHMWXLPE), is disposed within the socket 32 to reduce friction between the ball 22 and the socket and to increase the life of the joint. On the other hand, the socket insert 36 may comprise ceramic or metal or a ceramic or metal socket may be used without a socket insert. In some cases, the second load bearing surface in contact with a surface of a medical device component of cobalt-chromium comprises bone or bone cement. The convex outer surface of the ball 22 interfaces with the concave load bearing surface of the socket insert 36 or socket 32, as appropriate, to allow the joint to rotate and articulate simulating the movement of the natural hip joint.

A metal to metal interface, provided the surfaces are smooth and defect-free potentially offers the longest lifetime and, if the interfacing surfaces can be formed directly on the head and socket respectively, the largest area can be provided for the interface, thereby reducing the pressures between them.

For strength, corrosion resistance, and biocompatibility, the ball 22 comprises a cobalt-chromium alloy. Additional components of the prosthetic hip joint 20, including the body 24 and the socket 32 may also comprise a cobalt chromium alloy. Cobalt-chromium alloys are alloys comprising significant portions of cobalt and chromium and, commonly, also include a significant portion of molybdenum. Cobalt-chromium alloys used in medical devices are typified by alloys complying with ASTM standard specifications, ASTM F-75-01, STANDARD SPECIFICATION FOR COBALT-28 CHROMIUM-6 MOLYBDENUM ALLOY CASTINGS AND CASTING ALLOY FOR SURGICAL IMPLANTS, and ASTM-799, STANDARD SPECIFICATION FOR COBALT-28 CHROMIUM-6 MOLYBDENUM ALLOY FORGING FOR SURGICAL IMPLANTS.

Cobalt-chromium alloys also include alloys that have higher minor portions of carbon or nitrogen and comply with an ASTM F-75 Modified specification. In addition, as used herein, cobalt-chromium alloys include other proprietary alloys that contain cobalt and chromium and resemble alloys conforming to the ASTM-F75, modified ASTM-F75, and ASTM-799 standard specifications.

The ball head 22 and neck 26 are frequently separate components provided with a stem and socket interconnection (not shown), whereby the same ball head 22 can be applied to stems such as body 24, or to a stem fixed directly on the femur 30, or even to the bone itself. Moreover, different shapes and sizes of body 24 can be employed.

Cobalt-chromium, and more particularly cobalt-chromium-molybdenum, are desirably cast so that the microcrystalline structure is most favourable for the purposes of resisting corrosion, providing the smoothest possible surface and reducing any tendency to suffer fatigue cracks after an extended period of use within the patient's body. Moreover, the subsequent machining and welding operations should be minimised to reduce cost and also to reduce adverse consequences by inadvertent altering of the crystal structure.

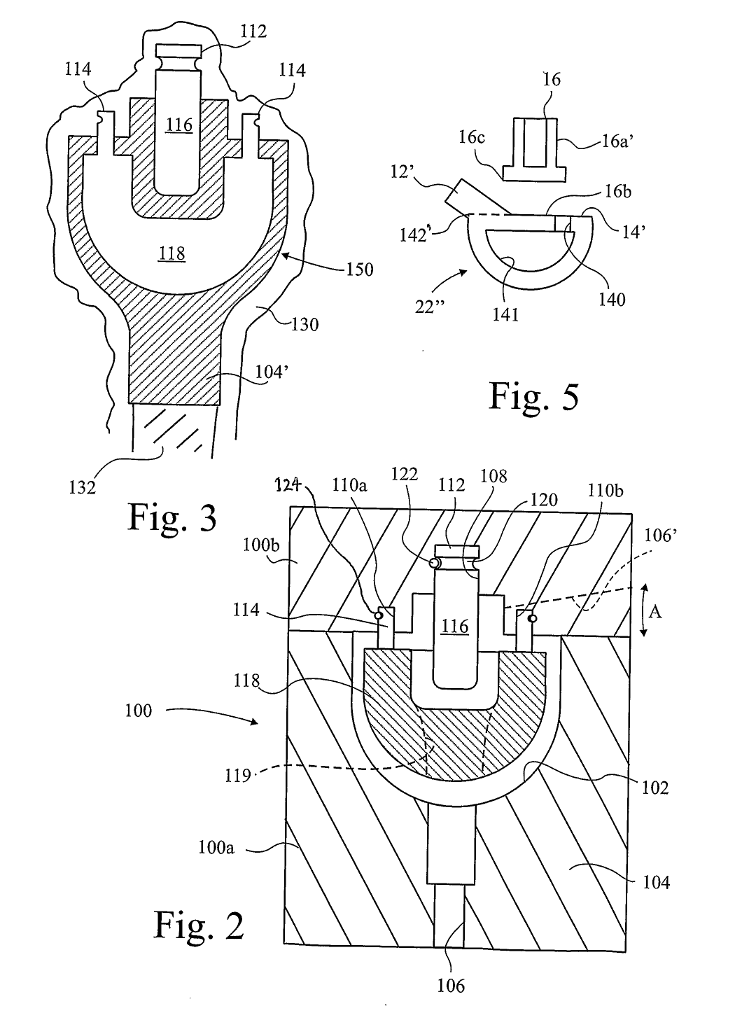

With reference to FIG. 4, a part completed head 22′ is shown which has a spherical (“domed”) surface 10 from which extends a gate 12, explained further below. A base surface 14 of the head 22′ has a central socket 16 to receive a plug (not shown) on the end of the neck 26 of the body 24. The head 22′ is formed in a casting process which begins (see FIG. 2) with the moulding of a wax pattern in an aluminium mould 100. The mould 100 is in two parts 100a,b, lower part 100a comprising a hemispherical former 102 having an integral gate-forming bore 104 through which a mould injection port 106 connects. The complimentary part 100b of the mould 100 includes blind bores 108,110a, b adapted to receive extensions 112,114 of cores 116,118 respectively.

Extension 112 of core 116 has a circumferential groove 120, which is engaged by a pin 122 that retains the core 116 in the bore 108 once the pin 122 is inserted from the side of the mould part 100b. Likewise, extensions 110a,b (typically there will be four of such extensions, only two of which are visible in FIG. 2) are also provided with grooves for engagement by pins 124 that retain the core 118 in engagement with the mould part 100b.

Once assembled as shown in FIG. 2, molten wax (or other suitable alternative) is injected through the port 106 to fill the void between the cores 116,118 and the mould parts 100a,b.

In FIG. 3, pins 122,124 have been removed and the mould parts 100a, 100b separated in the direction of the arrow A in FIG. 2 to expose a moulded wax pattern 150. The separation of the mould parts 10a,b occurs, of course, once the wax has solidified. The cores 116,118 are then captivated within the wax pattern 150.

As is well known in the art, a layer 130 of ceramic paste is applied to the outside of the patent 150 by sequentially dipping in powder and slurry, to build up a mould shell for metal casting. At this point, the wax that remained in injection port 106 has been removed and the gate pattern 104′ has been connected to a wax runner system 132 (not shown in detail) whereby a number of patterns 150 are connected together on a tree.

Once the shell 130 is built up, by consecutive dipping in slurry and powder, as is known in the art, the shell is allowed to dry and harden and subsequently is placed in a kiln. When the kiln is heated, the wax melts and is allowed to drain from the casting mould, leaving the cores 116,118 in place, retained in the shell 130 by their respective extensions 112,114 captured by the shell 130.

Once the wax has fully drained, the kiln is further heated to fire the ceramic shell and burn off any remaining hydrocarbon components of the wax.

Finally, the desired alloy is cast into the mould formed by the conjoined shells 130. Once the metal has solidified and cooled in an appropriate quenching cycle, the shell 130 is broken and removed and the gate 12 cut to separate the cast heads 22′ from the runner tree. The cores 116,118 are then removed, again by means known in the art. The core 116 can be withdrawn and, indeed, it is not necessarily ceramic and could be employed again in appropriate circumstances. However, the core 118 most likely is ceramic and of the type that shatters to a fine dust when vibrated with an appropriate tool. Once extensions 114 have been snapped off, a tool can be inserted through the apertures 140 so formed in the base 14 of the head 22′. The core 118 is then shattered and can be withdrawn through the apertures 140. Alternatively, the ceramic or alternative core material can be dissolved using an appropriate etching medium, again known in the art, for removal from the void within the head 22′. Such a medium may comprise a caustic solution such as potassium hydroxide. The apertures 140 need not be round, but could be elongate in a circumferential direction, as shown at 140′. This might facilitate removal of the core.

Once the void is empty of core, the apertures 140 are sealed by welding using the same material as the casting of the head 22′. Indeed, it is arranged that the apertures 140 are spaced from the edge 142 between the base 14 and domed surface 10. This ensures that the inevitable heating caused by the welding does not raise the temperature of the domed surface 10 to such an extent that the desirable microstructure is adversely affected. Alternatively, the core is not removed at all. This reduces the weight advantage of the present invention, but still reduces the cost compared with solid metal, and is still much less dense. Also, although weld filling is preferred, if a larger aperture 140,140′ is employed, it may be preferred to fit and weld in place a plug of the appropriate metal. Although the same metal as used in the casting is preferred, this is not essential.

Finally, once the apertures 140 are sealed, the gate 12 is removed so that the domed surface 10 can be formed with a perfect spherical surface, and any hardening process prior, to final polishing, can be effected. By this process, the required machining of the head 22′ can be reduced to perhaps nothing more than adjustment of the socket 16. Indeed, the socket 16 can be cast as a rough, somewhat tapered aperture, employing the ceramic shell to form it, in which event a simple machining operation can refine its dimensions.

Although the hollow interior of the head 22′ is isolated from the exterior it is quite possible to effect the sealing of the apertures 140 in an inert atmosphere, such as argon, so that it is that gas which fills the interior of the head in the unlikely event of any leakage therefrom in time.

The hollow-forming core 118 is shown as a single component. There is no reason why this should be essential. It could comprise several components spaced from one another. The resulting spaces between them would provide webs in the final product linking the trunnion 16a (as the socket-forming part of base is referred to) to the domed surface, thereby increasing the rigidity of the arrangement. Indeed, in FIG. 2, a column-forming bore 119 is shown in dotted lines in the core 118. This not only provides support for the trunnion 16a in the finished product, but also provides a better route for the molten metal as it enters the shell mould 130 and follows into the trunnion-forming part of the mould.

It is a feature of the present invention that at least a trunnion seat 16b (see FIG. 5), if not the trunnion 16a itself, is moulded integrally with the rest of the head. In FIG. 5, an alternative embodiment of the present invention provides only a trunnion seat 16b on the cast head 22″. The trunnion 16a′ is separately formed and subsequently welded to the trunnion seat. The trunnion 16a′ is shown as a cup with a weld flange 16c, but it could, of course, be a simple hollow cylinder. In this embodiment, the casting gate 12′ is shown inclined and connected to the base 14′ (formed by repositioned gate/port 106′ shown in dotted lines in FIG. 2 at the interface between mould parts 100a,b).

While separate formation of the trunnion is not especially preferred (because it reduces traceability of the components and increases the separate operations performed in the manufacture of the head, it still has the advantage that the welding edge 16c can be positioned well away from the edge 142′.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1. A method of casting a hollow medical prosthesis head comprising the steps of:
forming a pattern of the head, said pattern comprising a domed surface and a trunnion-seat-forming base, and a gate extension;
disposing a hollow-forming core in the head and an extension of said core through the base;
coating the pattern and said extension with a shell-forming slurry in which said core extension is captivated;
curing said shell to form a mould;
casting metal into the mould so as to form an aperture through the base of said head around said core extension;
when cool, removing said shell and at least said core extension; and
closing the aperture.

2. A method as claimed in claim 1, wherein said closure is effected by weld-filling of said aperture.

3. A method as claimed in claim 2, wherein said weld-filling uses the same metal as said cast metal.

4. A method as claimed in claim 1, wherein said closure is effected by welding a plug in said aperture.

5. A method as claimed in claim 1, wherein said pattern is moulded from settable fluid material.

6. A method as claimed in claim 5, wherein said material is wax and said curing step includes the step of melting the wax and draining said melted wax from the shell once the shell has cured to the extent that it retains its shape during said draining.

7. A method as claimed in claim 1, wherein there are a plurality of said extensions and apertures formed thereby.

8. A method as claimed in claim 1, wherein said gate extension is off said domed surface.

9. A method as claimed in claim 1, wherein said pattern includes a trunnion-forming part on said base and a trunnion is thereby integrally formed on the head during said casting step.

10. A method as claimed in claim 9, wherein a second, socket-forming core is disposed in said trunnion-forming part, said socket-forming core including an extension also captivated by said shell-forming slurry.

11. A method as claimed in claim 10, wherein said socket-forming core is ceramic.

12. A method as claimed in claim 1, wherein said hollow-forming core is toroidal in shape.

13. A method as claimed in claim 10, wherein said trunnion is formed by the eye of said toroid.

14. A method as claimed in claim 13, wherein said socket-forming core extends into the eye.

15. A method as claimed in claim 1, wherein said hollow-forming core is ceramic.

16. A method as claimed in claim 1, wherein all of said core is removed from the head prior to said closure.

17. A method as claimed in claim 16, wherein the hollow left by removal of said hollow-forming core is evacuated and/or filled with an inert gas before said closure is effected.

18. A method as claimed in claim 16, wherein said hollow-forming core is removed by shattering the core with a vibrating tool.

19. A method as claimed in claim 16, wherein said hollow-forming core is chemically etched to remove it.

20. A method as claimed in claim 1, wherein said extensions are elongate in section.

21. A method as claimed in claim 1, wherein said metal is a cobalt/chromium/molybdenum alloy.

22. A method as claimed in claim 5, wherein said moulding of the pattern comprises the steps of:
positioning said core or cores in a pattern-forming mould by connection of said extension or extensions to a base of said mould;
closing said mould with a domed cover; and
injecting curable fluid material the mould.

23. A method as claimed in claim 22, wherein said domed cover has a gate aperture forming said gate extension.

24. A method as claimed in claim 23, wherein said injection of said fluid material is through said gate aperture.

25. A method as claimed in claim 22, wherein said fluid material is molten wax.

26. A method as claimed in claim 1, in which said hollow medical prosthesis head is a hip prosthesis head.

27. A medical prosthesis head comprising a casting of metal provided with a domed surface and a base, and having an integrally cast hollow void, an integrally cast trunnion seat, and closed core pin aperture or apertures in the base of the head, said apertures being of smaller dimensions than the void.

28. A medical prosthesis head as claimed in claim 27, in which said core pin apertures are closed by weld-filling.

29. A medical prosthesis head as claimed in claim 27, wherein said trunnion seat includes an integrally cast trunnion.

30. A medical prosthesis head as claimed in claim 29, in which said trunnion includes an integrally cast socket.

31. A medical prosthesis head as claimed in claim 27, in which said trunnion seat is supported by an integrally cast column spanning said void and connected with said domed surface.

32. (canceled)
